(19) United States
(12) Patent Application Publication
(10) Pub. No.: US 2015/0164420 A1
(43) Pub. Date: Jun. 18, 2015

(54) STRUCTURE OF THREE-DIMENSIONAL ELECTRICALLY CONDUCTIVE FABRIC

(71) Applicant: KING'S METAL FIBER TECHNOLOGIES CO., LTD., Taichung City (TW)

(72) Inventors: HONG-HSU HUANG, TAIPEI CITY (TW); I-CHEN SU, TAIPEI CITY (TW); KING-MU HSIAO, TAIPEI CITY (TW); SHUN-TUNG YANG, TAIPEI CITY (TW)

(21) Appl. No.: 14/630,970

(22) Filed: Feb. 25, 2015

Related U.S. Application Data
(63) Continuation-in-part of application No. 13/775,421, filed on Feb. 25, 2013.

(51) Int. Cl. A61B 5/00; A61B 5/04; H01B 1/20; D04B 1/14; D04B 1/18; D02G 3/44

(52) U.S. Cl. CPC A61B 5/6804; D04B 1/18; D02G 3/441; H01B 1/20; D04B 1/14; A61B 5/04; D10B 2509/00; D10B 2401/16

(57) ABSTRACT

A structure of three-dimensional electrically conductive fabric includes a resilient conductive tissue, a foundation tissue, and a support tissue. The support tissue is arranged between and connects the resilient conductive tissue and the foundation tissue. The resilient conductive tissue, the foundation tissue, and the support tissue are unitarily combined through knitting to form the structure of three-dimensional electrically conductive fabric.

### REFERENCE TO RELATED APPLICATION

This Application is being filed as a Continuation-in-Part of application Ser. No. 13/775,421, filed 25 Feb. 2013, currently pending.

### FIELD OF THE INVENTION

The present invention relates to a structure of three-dimensional electrically conductive fabric, and in particular to a structure of three-dimensional electrically conductive fabric that features both resiliency and electrical conductivity.

### BACKGROUND OF THE INVENTION

As shown in FIG. 1, a conventional detection element 1 for physiological examination comprises a base layer 10 and an electrically conductive layer 11 formed on the base layer 10. To use, the electrically conductive layer is attached to human skin surface to detect a signal generated by the human body. However, the electrically conductive 11 of such a detection element 1 is generally of poor resiliency and has poor electrical conductivity with human skin is poor, making it difficult to detect the signal generated by the human body and also making wear uncomfortable. As shown in FIG. 2, an improvement is made such that a resilient layer 12 is arranged between the electrically conductive layer 11 and the base layer 10 so that contact tightness between the electrically conductive layer 11 and human skin can be improved with the resilient layer 12. Further, a moisture-retaining material is also included in the layer to make the layer also function moisture retaining thereby improving electrical conductivity of the electrically conductive layer 11. However, since the resilient layer 12 and the electrically conductive layer 11 are two separate layers, moisture must penetrate through the electrically conductive layer 11 before being absorbed by the resilient layer 12. Consequently, the absorbability of moisture is affected. When the resilient layer 12 releases water between the electrically conductive layer 11 and human skin, the release of water is also affected by being blocked by the electrically conductive layer 11. Further, since the resilient layer 12 and the electrically conductive layer 11 are two separate layers that are bonded to each other by an external force (such as adhesion). These layers are easily detached from each other due to the high humidity long maintained by the resilient layer 12, making the detection element 1 losing its function.

In view of this problem, the present invention aims to provide a structure that possesses the characteristics of resiliency, electrical conduction, and moisture retention in order to achieve the goal of improving electrical conduction and lifespan of product.

### SUMMARY OF THE INVENTION

An object of the present invention is to provide a structure of three-dimensional electrically conductive fabric that is formed through being unitarily knitted and features resiliency and electrical conductivity.

Another object of the present invention is to provide a structure of three-dimensional electrically conductive fabric that features moisture retention.

To realize the above objects, the present invention provides a structure of three-dimensional electrically conductive fabric, which comprises a resilient conductive tissue, which is formed by arranging and interlacing a plurality of first structural yarns, a plurality of second structural yarns, a plurality of first elastic yarns, a plurality of second elastic yarns, and a plurality of electrically conductive yarns along first dimension and second dimension, wherein each of the first structural yarns is combined with each of the first elastic yarns as a first strand, each of the second structural yarns is combined with each of the second elastic yarns as a second strand and a plurality of first stitches are formed by individually interlocking each of the first strand and each of the second strand along the second dimension; a foundation tissue, which is formed by arranging and interlacing a plurality of third structural yarns, a plurality of fourth structural yarns, a plurality of fifth structural yarns, a plurality of third elastic yarns, a plurality of fourth elastic yarns and a plurality of fifth elastic yarns along the first dimension and the second dimension, wherein each of the third structural yarns is arranged with each of the third elastic yarns as a third strand, each of the fourth structural yarns is arranged with each of the fourth elastic yarns as a fourth strand, each of the fifth structural yarns 400C is arranged with each of the fifth elastic yarns as a fifth strand and a plurality of second stitches are formed by individually interlocking each of the third strand and each of the fourth strand along the second dimension; and a support tissue, which is formed of a plurality of first support yarns and a plurality of second support yarns and connects between the resilient conductive tissue and the foundation tissue, wherein each of the first support yarns is interlocking with the plurality of first stitches along the second dimension and extends to the foundation tissue along third dimension to be interlocked with the plurality of second stitches along the second dimension, and a plurality of third stitches are formed by individually interlocking each of the second support yarns with each of the fifth strand along the second dimension and the second support yarns extends to the resilient conductive tissue along the third dimension to form a plurality of fourth stitches by interlocking the second support yarns with the plurality of electrically conductive yarns along the second dimension, wherein the plurality of third stitches individually space from the plurality of fourth stitches along the first dimension, the plurality of fourth stitches individually space from the plurality of first stitches along the first dimension and the electrically conductive yarns project beyond a surface of the resilient conductive tissue.

In the above-discussed structure of three-dimensional electrically conductive fabric, the first structural yarns, the second structural yarns, the third structural yarns, the fourth structural yarns and the fifth structural yarns are each one of polyester yarn, porous fiber yarn, alginate fiber yarn, carboxymethyl cellulose fiber yarn, and rayon fiber yarn.

In the above-discussed structure of three-dimensional electrically conductive fabric, the electrically conductive yarns are one of metal fiber yarn, carbon nanotube fiber yarn, and carbon fiber yarn.

In the above-discussed structure of three-dimensional electrically conductive fabric, the first elastic yarns, the second elastic yarns, the third elastic yarns, the fourth elastic yarns and the fifth elastic yarns are each spandex yarn.

In the above-discussed structure of three-dimensional electrically conductive fabric, the first support yarns and the second support yarns are each one of polyester yarn and nylon yarn.

In the above-discussed structure of three-dimensional electrically conductive fabric, the first structural yarns, the second structural yarns, the first elastic yarns, the second elastic yarns and the electrically conductive yarns are arranged and interlaced through knitting to form the resilient conductive tissue.

In the above-discussed structure of three-dimensional electrically conductive fabric, the third structural yarns, the fourth structural yarns, the fifth structural yarns, the third elastic yarns, the fourth elastic yarns and the fifth elastic yarns are arranged and interlaced through knitting to form the foundation tissue.

In the above-discussed structure of three-dimensional electrically conductive fabric, the resilient conductive tissue, the foundation tissue, and the support tissue are unitarily combined to form the structure of three-dimensional electrically conductive fabric, in which the same planar tissue features both resiliency and electrical conductivity and also shows an effect of moisture retention through being combined with structural yarns that feature moisture retention.

### BRIEF DESCRIPTION OF THE DRAWINGS

The present invention will be apparent to those skilled in the art by reading the following description of preferred embodiments thereof with reference to the drawings, in which:

FIG. 1 is a side elevational view showing a conventional detection element for physiological examination;

FIG. 2 is a side elevational view showing a conventional detection element for physiological examination;

FIG. 3 is a schematic view showing a structure of three-dimensional electrically conductive fabric according to the present invention;

FIG. 4 is a perspective view showing, in an enlarged form, a portion of the structure of three-dimensional electrically conductive fabric in accordance with the present invention; and

FIG. 5 is a cross-sectional view showing, in an enlarged form, a portion of the structure of three-dimensional electrically conductive fabric in accordance with the present invention.

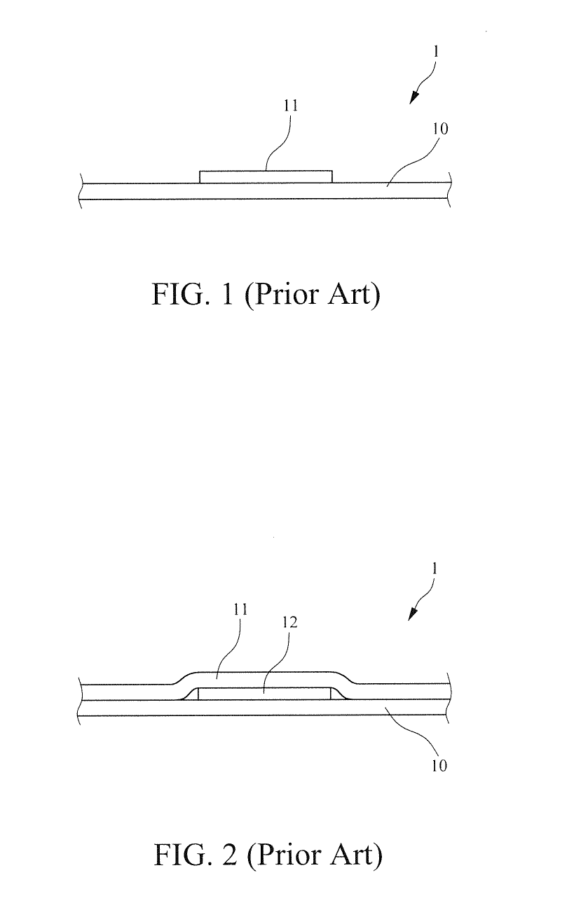

### DETAILED DESCRIPTION OF THE PREFERRED EMBODIMENTS

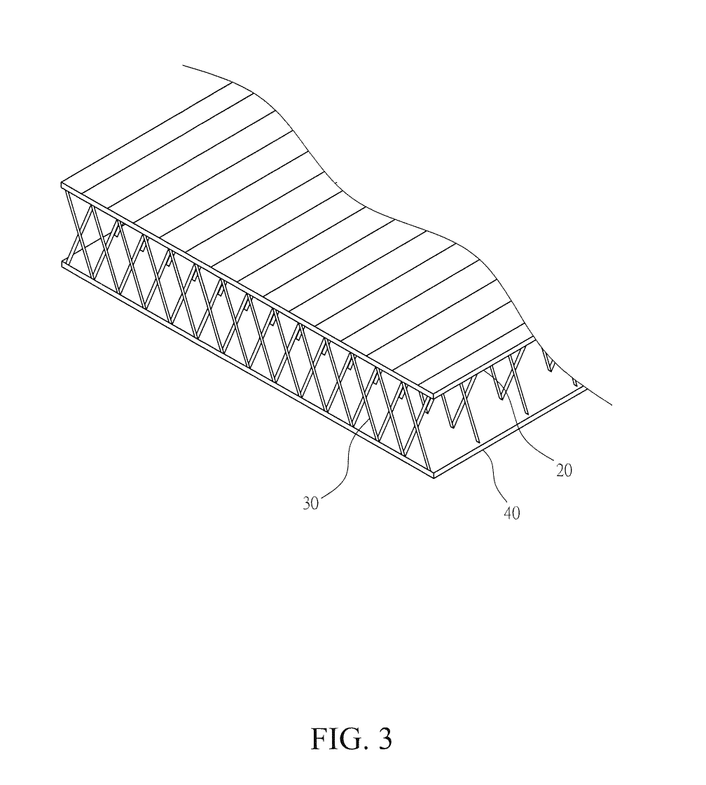

With reference to the drawings and in particular to FIG. 3, which is a perspective view showing a structure of three-dimensional electrically conductive fabric according to the present invention, as shown in the drawing, in the instant embodiment, the structure of three-dimensional electrically conductive fabric according to the present invention comprises a resilient conductive tissue 20, a support tissue 30, and a foundation tissue 40, which are knitted unitarily to form the structure of three-dimensional electrically conductive fabric with the support tissue 30 arranged between and connecting the resilient conductive tissue 20 and the foundation tissue 40.

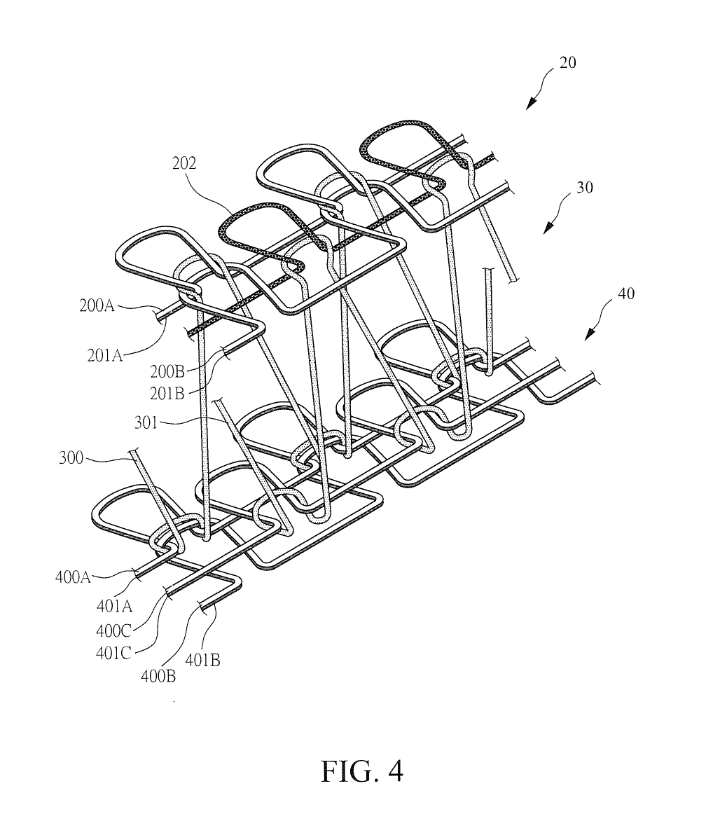

Referring to FIG. 4, which is a perspective view showing, in an enlarged form, a portion of the structure of three-dimensional electrically conductive fabric in accordance with the present invention, as shown in the drawing, the resilient conductive tissue 20 is formed by arranging and interlacing a plurality of first structural yarns 200A, a plurality of second structural yarns 200B, a plurality of first elastic yarns 201A, a plurality of second elastic yarns 201B, and a plurality of electrically conductive yarns 202 along first dimension and second dimension, wherein each of the first structural yarns 200A is combined with each of the first elastic yarns 201A as a first strand, each of the second structural yarns 200B is combined with each of the second elastic yarns 201B as a second strand and a plurality of first stitches are formed by individually interlocking each of the first strand and each of the second strand along the second dimension.

A foundation tissue 40 is formed by arranging and interlacing a plurality of third structural yarns 400A, a plurality of fourth structural yarns 400B, a plurality of fifth structural yarns 400C, a plurality of third elastic yarns 401A, a plurality of fourth elastic yarns 401B and a plurality of fifth elastic yarns 401C along the first dimension and the second dimension, wherein each of the third structural yarns 400A is arranged with each of the third elastic yarns 401A as a third strand, each of the fourth structural yarns 400B is arranged with each of the fourth elastic yarns 401B as a fourth strand, each of the fifth structural yarns 400C is arranged with each of the fifth elastic yarns 401C as a fifth strand and a plurality of second stitches are formed by individually interlocking each of the third strand and each of the fourth strand along the second dimension.

A support tissue 30 is formed of a plurality of first support yarns 300 and a plurality of second support yarns 301 and connects between the resilient conductive tissue 20 and the foundation tissue 40, wherein each of the first support yarns 300 is interlocking with the plurality of first stitches along the second dimension and extends to the foundation tissue 40 along third dimension to be interlocked with the plurality of second stitches along the second dimension, and a plurality of third stitches are formed by individually interlocking each of the second support yarns 301 with each of the fifth strand along the second dimension and the second support yarns 301 extends to the resilient conductive tissue 20 along the third dimension to form a plurality of fourth stitches by interlocking the second support yarns 301 with the plurality of electrically conductive yarns 202 along the second dimension, wherein the plurality of third stitches individually space from the plurality of fourth stitches along the first dimension, the plurality of fourth stitches individually space from the plurality of first stitches along the first dimension and the electrically conductive yarns 202 project beyond a surface of the resilient conductive tissue 20. The interlaced arrangement of the first support yarns 300 and the second support yarns 301 provides improved resiliency to the structure of three-dimensional electrically conductive fabric of the present invention, so as to make a wearer comfortable when is used to make a wearable article. Further, the first support yarns 300 and the second support yarns 301 form tiny voids therebetween that help retaining moisture and improving electrical conductivity.

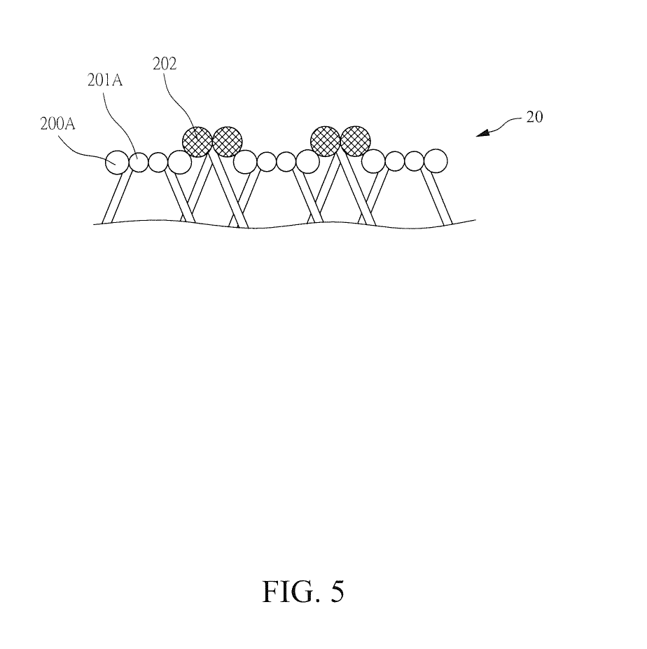

Referring to FIG. 5, which is a cross-sectional view showing, in an enlarged form, a portion of the structure of three-dimensional electrically conductive fabric in accordance with the present invention, as shown in drawing, the resilient conductive tissue 20 is formed by arranging and interlacing a plurality of first structural yarns 200A, a plurality of first elastic yarns 201A, and a plurality of electrically conductive yarns 202 together. Each of the first structural yarns 200A is combined with each of the first elastic yarns 201A as a strand for being arranged alternately with each of the electrically conductive yarns 202, whereby after the entirety of the structure of three-dimensional electrically conductive fabric is completely arranged when the stretching force of yarns are removed, the first elastic yarns 201A get contracting and squeeze the electrically conductive yarns 202 outward so that the electrically conductive yarns 202 project beyond the surface of the entire resilient conductive tissue 20. This ensures that when the fabric is placed on human body, the electrically conductive yarns 202 get contact with the human body first so that the structure of three-dimensional electrically conductive fabric according to the present invention may provide improved effect of detection.

The first structural yarns 200A, the second structural yarns 200B, the third structural yarns 400A, the fourth structural yarns 400B and the fifth structural yarns 400C can selectively be one of polyester yarn, porous fiber yarn, alginate fiber yarn, carboxymethyl cellulose fiber yarn, and rayon fiber yarn, among which porous fiber yarn, alginate fiber yarn, carboxymethyl cellulose fiber yarn, and rayon fiber yarn have the function of moisture retention. If the first structural yarns 200A, the second structural yarns 200B, the third structural yarns 400A, the fourth structural yarns 400B and the fifth structural yarns 4000 are selected from these four materials, then the structure of three-dimensional electrically conductive fabric according to the present invention may shows the characteristics of resiliency, moisture retention, and electrical conductivity.

The first elastic yarns 201A, the second elastic yarns 201B, the third elastic yarns 401A, the fourth elastic yarns 401 B and the fifth elastic yarns 401C can be spandex yarn. The electrically conductive yarns 202 can selectively be one of metal fiber yarn, carbon nanotube fiber yarn, and carbon fiber yarn. The first support yarns 300 and the second support yarns 301 can selectively be one of polyester yarn and nylon yarn.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A structure of three-dimensional electrically conductive fabric, comprising:
a resilient conductive tissue, which is formed by arranging and interlacing a plurality of first structural yarns, a plurality of second structural yarns, a plurality of first elastic yarns, a plurality of second elastic yarns, and a plurality of electrically conductive yarns along first dimension and second dimension, wherein each of the first structural yarns is combined with each of the first elastic yarns as a first strand, each of the second structural yarns is combined with each of the second elastic yarns as a second strand and a plurality of first stitches are formed by individually interlocking each of the first strand and each of the second strand along the second dimension;
a foundation tissue, which is formed by arranging and interlacing a plurality of third structural yarns, a plurality of fourth structural yarns, a plurality of fifth structural yarns, a plurality of third elastic yarns, a plurality of fourth elastic yarns and a plurality of fifth elastic yarns along the first dimension and the second dimension, wherein each of the third structural yarns being arranged with each of the third elastic yarns as a third strand, each of the fourth structural yarns being arranged with each of the fourth elastic yarns as a fourth strand, each of the fifth structural yarns being arranged with each of the fifth elastic yarns as a fifth strand and a plurality of second stitches are formed by individually interlocking each of the third strand and each of the fourth strand along the second dimension; and
a support tissue, which is formed of a plurality of first support yarns and a plurality of second support yarns and connects between the resilient conductive tissue and the foundation tissue, wherein each of the first support yarns is interlocking with the plurality of first stitches along the second dimension and extends to the foundation tissue along third dimension to be interlocked with the plurality of second stitches along the second dimension, and a plurality of third stitches are formed by individually interlocking each of the second support yarns with each of the fifth strand along the second dimension and the second support yarns extends to the resilient conductive tissue along the third dimension to form a plurality of fourth stitches by interlocking the second support yarns with the plurality of electrically conductive yarns along the second dimension, wherein the plurality of second stitches individually space from the plurality of third stitches along the first dimension, the plurality of first stitches individually space from the plurality of fourth stitches along the first dimension and the electrically conductive yarns project beyond a surface of the resilient conductive tissue.

2. The structure of three-dimensional electrically conductive fabric as claimed in claim 1, wherein the first structural yarns, the second structural yarns, the third structural yarns, the fourth structural yarns and the fifth structural yarns are each one of polyester yarn, porous fiber yarn, alginate fiber yarn, carboxymethyl cellulose fiber yarn, and rayon fiber yarn.

3. The structure of three-dimensional electrically conductive fabric as claimed in claim 1, wherein the electrically conductive yarns are one of metal fiber yarn, carbon nanotube fiber yarn, and carbon fiber yarn.

4. The structure of three-dimensional electrically conductive fabric as claimed in claim 1, wherein the first elastic yarns, the second elastic yarns, the third elastic yarns, the fourth elastic yarns and the fifth elastic yarns are each spandex yarn.

5. The structure of three-dimensional electrically conductive fabric as claimed in claim 1, wherein the first support yarns and the second support yarns are each one of polyester yarn and nylon yarn.

6. The structure of three-dimensional electrically conductive fabric as claimed in claim 1, wherein the first structural yarns, the second structural yarns, the first elastic yarns, the second elastic yarns and the electrically conductive yarns are arranged and interlaced through knitting to form the resilient conductive tissue.

7. The structure of three-dimensional electrically conductive fabric as claimed in claim 1, wherein the third structural yarns, the fourth structural yarns, the fifth structural yarns, the third elastic yarns, the fourth elastic yarns and the fifth elastic yarns are arranged and interlaced through knitting to form the foundation tissue.
